SEQUENCE LISTING (1 sequence)

SEQ ID NO: 1 - protein, 9 aa, artificial sequence
  Synthetic substrate for SdeA ubiquitination assay
  Met (1)..(1): N-terminal fluorescein
  Asp (9)..(9): C-terminal amidation
MSSMNPEYD

(19) United States
(12) Patent Application Publication
(10) Pub. No.: US 2021/0396743 A1
(43) Pub. Date: Dec. 23, 2021

(54) FLUORESCENT ASSAY SYSTEMS FOR REAL-TIME MEASUREMENT OF PROTEIN UBIQUITINATION AND USES THEREOF

(71) Applicant: Purdue Research Foundation, West Lafayette, IN (US)

(72) Inventors: Chittaranjan Das, West Lafayette, IN (US); Kedar Puvar, West Lafayette, IN (US); Aya M. Saleh, West Lafayette, IN (US); Ryan William Curtis, Lafayette, IN (US); Tamara Lea Kinzer-Ursem, West Lafayette, IN (US); Jean Anne Chmielewski, Lafayette, IN (US)

(73) Assignee: Purdue Research Foundation, West Lafayette, IN (US)

(21) Appl. No.: 17/351,422

(22) Filed: Jun. 18, 2021

Related U.S. Application Data
(60) Provisional application No. 63/040,592, filed on Jun. 18, 2020.

(51) Int. Cl. G01N 33/544; G01N 21/64; G01N 33/68

(52) U.S. Cl. CPC G01N 33/544; G01N 2021/6439; G01N 33/6845; G01N 21/6428

(57) ABSTRACT

A radical departure from the classical E1-E2-E3 three-enzyme mediated ubiquitination of eukaryotes, the bacterial enzymes of the SidE family of Legionella pneumophila effectors utilize NAD⁺ to ligate ubiquitin onto target substrate proteins achieved via a two-step mechanism involving (1) ADP-ribosylation of ubiquitin followed by (2) phosphotransfer to a target serine residue. Using fluorescent NAD⁺ analogues as well as synthetic substrate mimics, a continuous assay system enabling real-time monitoring of both steps of this mechanism is disclosed herein. These assays are amenable to biochemical studies and high-throughput screening of inhibitors of these effectors, and enable the discovery and characterization of putative enzymes similar to the SidE family in other organisms. A kit of the assay system, for real-time monitoring protein ubiquitination and/or identifying an inhibitor for protein ubiquitination comprising a fluorescent NAD+ analogue and a synthetic substrate mimic, is also in the scope of this disclosure.

### CROSS-REFERENCE TO RELATED APPLICATIONS

The present U.S. patent application is related to and claims the priority benefit of U.S. Provisional Patent Application Ser. No. 63/040,592, filed Jun. 18, 2020, the contents of which are hereby incorporated by reference in their entirety into the present disclosure.

### GOVERNMENT SUPPORT CLAUSE

This invention was made with government support under GM126296 awarded by the National Institutes of Health. The government has certain rights in the invention.

### STATEMENT OF SEQUENCE LISTING

A computer-readable form (CRF) of the Sequence Listing is submitted with this application. The file generated on May 27, 2021 is entitled 69098-02_Seq_Listing_ST25_txt, the contents of which are incorporated herein in their entirety. Applicant states that the content of the computer-readable form is the same and the information recorded in computer readable form is identical to the written sequence listing.

### TECHNICAL FIELD

The present application relates to an assay system, or a kit thereof, for real-time monitoring protein ubiquitination and/or identifying an inhibitor for protein ubiquitination comprising a fluorescent NAD⁺ analogue and a synthetic substrate mimic.

### BACKGROUND INFORMATION

This section introduces aspects that may help facilitate a better understanding of the disclosure. Accordingly, these statements are to be read in this light and are not to be understood as admissions about what is or is not prior art.

The post-translational modification of proteins with the 76-residue modifier ubiquitin (Ub) is a vital process in eukaryotic cells.′ Ubiquitination, catalyzed by the three-enzyme E1-E2-E3 cascade and using ATP as a cofactor, is involved in protein recycling, DNA repair, and immunity. Most eukaryotic proteins undergo this modification at some point in time, highlighting its importance in proper cellular function. A rapidly-evolving area of research pertains to the interactions of bacterial effectors with the host ubiquitin system.² Prokaryotic organisms notably lack ubiquitin systems. However, several pathogenic bacteria have evolved a variety of strategies to effect the ubiquitin signaling of their eukaryotic hosts, such as mimics of eukaryotic ubiquitin-interacting proteins as well as novel motifs of their own.³ A striking example of the latter is the unusual NAD⁺-dependent ubiquitination of substrates by the SidE family of L. pneumophila effectors. The SidE family is comprised of four large (>150 kDa) modular proteins: SdeA, SdeB, SdeC, and SidE. During infection, these and other proteins are translocated into the host cell by way of the Dot/Icm type IV secretion system of the bacteria. These proteins are noteworthy in that they have been found to carry out the first example of protein ubiquitination occurring via a mechanism outside of the canonical eukaryotic pathway.⁴ This process, using NAD⁺ instead of ATP and requiring only one protein instead of three, results in a ribose-phosphate linkage between Arg42 of Ub and a Ser residue of the substrate.^{5,6} This stands in contrast to the standard isopeptide linkage between the C-terminus of Ub and a Lys residue of the substrate observed in typical ubiquitination.

Along with this ubiquitin-ligating activity, the SidE proteins are able to generate phosphoribosylated ubiquitin as a byproduct (Ub-PR).

Mechanistically, this ubiquitination reaction is initiated by a mono ADP-ribosyltransferase (mART) reaction of Arg42 of Ub (catalyzed by the mART domain), resulting in the intermediate Ub-ADP-ribose (Ub-ADPR). This molecule undergoes a subsequent phosphotransferase reaction (catalyzed by the phosphodiesterase, PDE, domain) to be linked onto a substrate Ser (and possibly other hydroxyl-containing) residue or simply to be hydrolyzed into Ub-PR, mechanistically explained as a phosphotransfer to water (FIG. 1).⁷ Interestingly, another Legionella effector, SidJ, has been found to be able to block SidE activity by glutamylating their catalytic residues, acting as a regulator of this process.⁸⁻¹¹ Recently, two other enzymes, homologous to the PDE domain, called DupA and DupB, were also found to regulate SidE-catalyzed ubiquitination. Instead of blocking catalysis, however, they remove phosphoribosyl-linked Ub from substrate proteins, reversing the modification.^{12,13} These two regulatory mechanisms highlight a remarkable complexity to this new post-translational modification.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIGS. 1A-1B show the general features of SdeA. (FIG. 1A) Three key constructs were utilized in this study: full length enzyme, a construct containing both PDE and mART domains, and a construct containing only the mART domain. (FIG. 1B) Overall SidE mechanism of action that involves first mono-ADP ribosylation of ubiquitin at Arg42, catalyzed by the mART domain. Next, this ADP-ribosylated intermediate reacts with the PDE domain to be transferred to a Ser residue of the substrate protein.

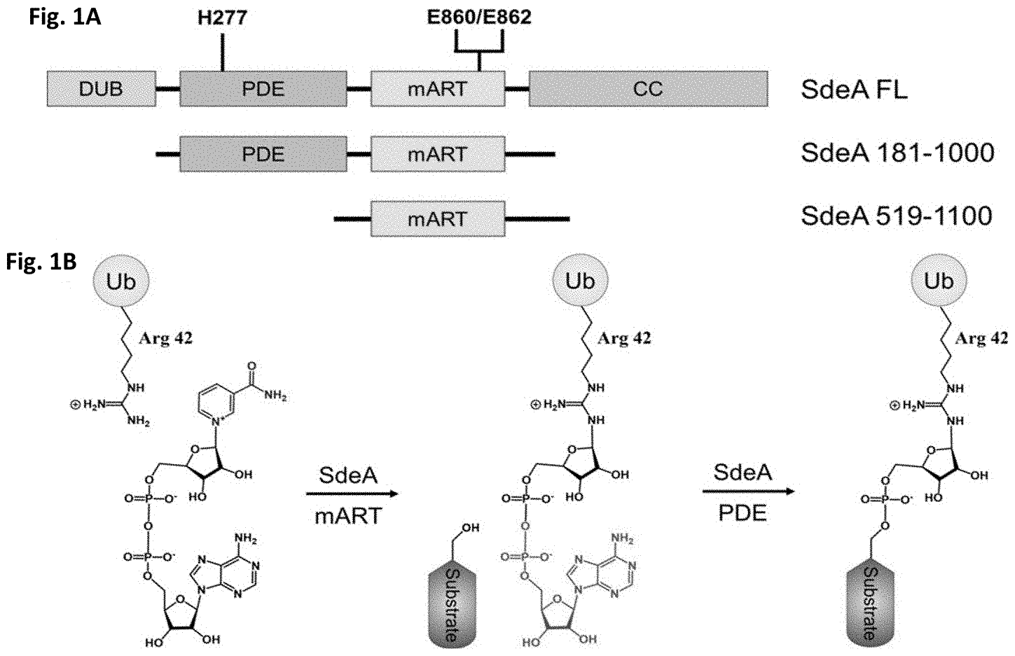

FIGS. 2A-2B depict fluorescent NAD⁺ analogues used for monitoring the first step of SdeA-catalyzed ubiquitination. (FIG. 2A) Structures of NAD⁺, εNAD⁺, and N^{tz}AD⁺. (FIG. 2B) When SdeA and Ub are incubated with εNAD⁺, a marked increase in fluorescence is observed in a time-dependent manner, consistent with the liberation of the nicotinamide group and loss of quenching. The activities of three distinct constructs of SdeA are compared.

FIG. 3. Structure of the synthetic peptide substrate for SdeA ubiquitination assays (Fluorescein-Met-Ser-Ser-Met-Asn-Pro-Glu-Tyr-Asp-amide, SEQ ID NO: 1). A nine-residue peptide with an N-terminal fluorescein group was designed, derived from the N-terminus of the known ubiquitination substrate protein Rab1.

FIG. 4A shows the overall scheme of a ubiquitination assay. FIG. 4B depicts fluorescence imaging of SDS-PAGE analysis of SdeA reaction in the presence of the peptide substrate reveals a fluorescent band when NAD⁺ is included, indicative of successful reaction. FIG. 4C is the crystal structure of SdeA mART and PDE domains showing interdomain interactions, the importance of which can be tested via this fluorometric assay (Protein Data Bank entry 5ZQ2).(18) FIG. 4D shows that including the fluorescent Rab1 peptide in the SdeA reaction caused a significant increase in FP. FIG. 4E shows selective mutation of the Ser residues in the peptide substrate shows that the third Ser is likely targeted. FIG. 4F shows mutation of the Ser to other hydroxyl-containing residues, such as Thr or Tyr, causes activity to be lost, indicating that SdeA specifically targets Ser residues. FIG. 4G shows the analysis of SdeA mutants using the fluorometric assay allows us to compare their activity in real time. The catalytic mutants (E/A and H/A) as well as the intradomain binding mutant (SdeA LF/D) were utilized. FIG. 4H shows the analysis of inhibition of SdeA-catalyzed ubiquitination. The nucleotides ADPR and AMP were included in the reaction mixture, resulting in inhibition of peptide ubiquitination. FIG. 4I demonstrates that substituting cell lysates for purified SdeA protein allows this assay to be used to detect ubiquitination of the synthetic peptide. The L. pneumophila lysate caused an increase in the level of polarization. Within the parameters of this assay, the HEK cell lysate did not show a detectable increase.

FIG. 5A shows the analysis of SidE regulators. SidJ was preincubated with SdeA for the indicated time points, and ubiquitination was measured. FIG. 5B shows the purified DupB (SdeD) was added to the preubiquitinated peptide, demonstrating removal of PR-linked Ub.

FIG. 5C demonstrates that L. pneumophila lysate without SidEs was also utilized in the two-step assay described above to show Dup activity.

FIG. 6 shows reaction profile of SdeA incubated with NtzAD+. This fluorescent NAD+ derivative exhibits fluorescence as its nicotinamide group is released similarly to εNAD+. We observed a lower fold increase in fluorescence intensity, in line with the differences in quantum yield increase upon nicotinamide cleavage reported for NtzAD+ and εNAD+.

FIGS. 7A-7C show the identification of fluorescent SdeA reaction products. (FIG. 7A) LC-MS of Ub-eADPR isolated from reaction mixture. (FIG. 7B) MALDI spectrum of ubiquitinated Rab1 peptide. (FIG. 7C) Comparison of ubiquitin-modifying activity of SdeA using either NAD+ or εNAD+. Ub was incubated with SdeA181-1000 H284A with either NAD+, εNAD+, or no nucleotide, and reactions were subjected to native-PAGE gel analysis, where the modified Ub-ADPR migrated farther due to the charge difference.

FIGS. 8A-8D depict analysis of SidE regulators. (FIG. 8A) Scheme of glutamylation of Glu862 by the calmodulin-activated effector SidJ. (FIG. 8B) Progress curve of SidJ activity determined by plotting initial rate of SdeA reactions, monitored by FP assay, versus length of time SdeA samples were reacted with SidJ. (FIG. 8C) Scheme of deconjugation of phosphoribosyl-linked ubiquitin from substrates by Dup enzymes. (FIG. 8D) SDS-PAGE showing ubiquitinated fluorescent peptide formation upon incubation with SdeA and NAD+. This ubiquitinated peptide band disappears when DupB is included, consistent with its reported deconjugating activity.

The attached drawings are for purposes of illustration and are not necessarily to scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

In some illustrative embodiments, this disclosure relates to an assay system for real-time monitoring protein ubiquitination and/or for identifying an inhibitor for protein ubiquitination comprising
a. a fluorescent NAD⁺ analogue;
b. a synthetic substrate mimic or a target compound as said inhibitor;
c. an enzyme of the SidE family for catalyzing ubiquitination.

In some illustrative embodiments, this disclosure relates to an assay system for real-time monitoring protein ubiquitination and/or for identifying an inhibitor for protein ubiquitination as disclosed herein further comprising 50 mM Tris pH 7.4, 100 mM NaCl, 1 mg/mL BSA, and NAD⁺ as a control.

In some illustrative embodiments, this disclosure relates to an assay system for real-time monitoring protein ubiquitination and/or for identifying an inhibitor for protein ubiquitination as disclosed herein, wherein said fluorescent NAD⁺ analogue is

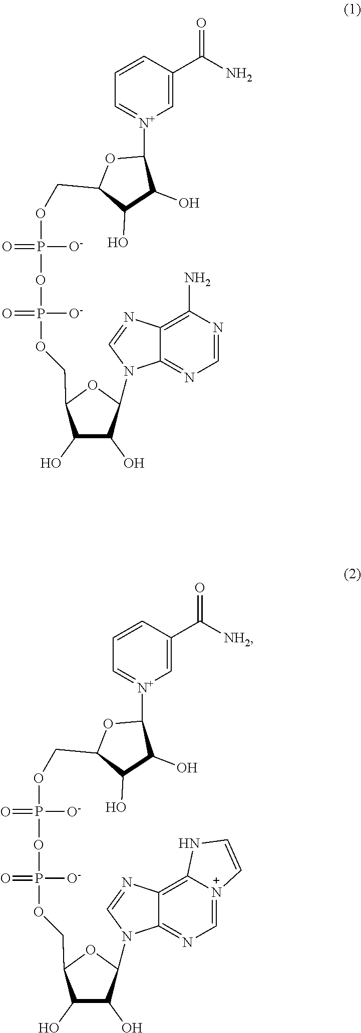

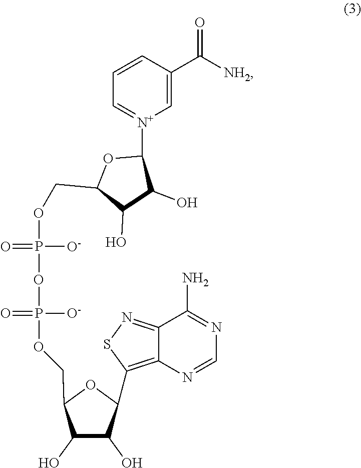

or a salt thereof.

In some illustrative embodiments, this disclosure relates to an assay system for real-time monitoring protein ubiquitination and/or for identifying an inhibitor for protein ubiquitination as disclosed herein, wherein said synthetic substrate mimic is

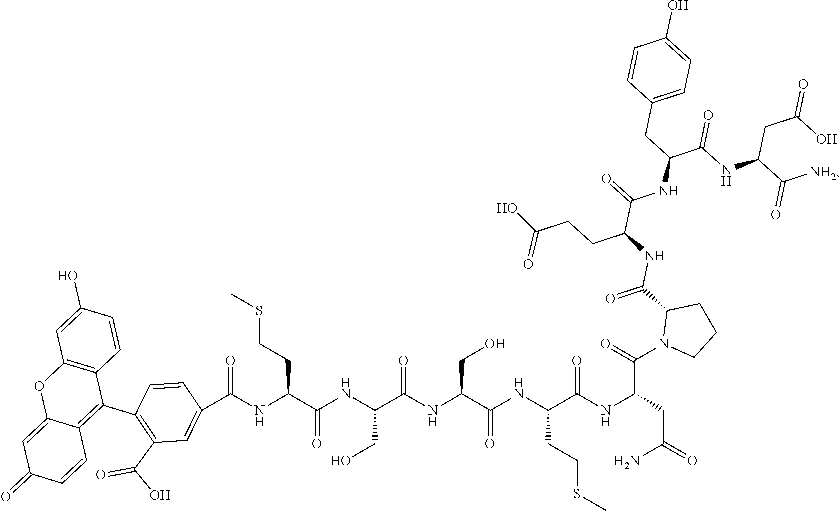 [SMILES: CSCC[C@H](NC(=O)c1ccc(-c2c3ccc(=O)cc-3oc3cc(O)ccc23)c(C(=O)O)c1)C(=O)N[C@@H](CO)C(=O)N[C@@H](CO)C(=O)N[C@@H](CCSC)C(=O)N[C@@H](CC(N)=O)C(=O)N1CCC[C@H]1C(=O)N[C@@H](CCC(=O)O)C(=O)N[C@@H](Cc1ccc(O)cc1)C(=O)N[C@@H](CC(=O)O)C(N)=O]

or a salt thereof (SEQ ID NO: 1).

In some illustrative embodiments, this disclosure relates to an assay system for real-time monitoring protein ubiquitination and/or for identifying an inhibitor for protein ubiquitination as disclosed herein, wherein said assay system is configured for high-throughput screening for ubiquitination inhibitors.

In some illustrative embodiments, this disclosure relates to an assay system for real-time monitoring protein ubiquitination and/or for identifying an inhibitor for protein ubiquitination as disclosed herein, wherein said assay system is configured for monitoring the reverse process of protein ubiquitination.

In some illustrative embodiments, this disclosure relates to an assay kit for real-time monitoring protein ubiquitination and/or for identifying an inhibitor for protein ubiquitination comprising
a. a fluorescent NAD⁺ analogue;
b. a synthetic substrate mimic or a compound as said inhibitor for protein ubiquitination; and
c. an enzyme of the SidE family for catalyzing ubiquitination.

In some illustrative embodiments, this disclosure relates to an assay kit for real-time monitoring protein ubiquitination and/or for identifying an inhibitor for protein ubiquitination as disclosed herein further comprising 50 mM Tris pH 7.4, 100 mM NaCl, 1 mg/mL BSA, and NAD+ as a control.

In some illustrative embodiments, this disclosure relates to an assay kit for real-time monitoring protein ubiquitination and/or for identifying an inhibitor for protein ubiquitination as disclosed herein, wherein said fluorescent NAD+ analogue is

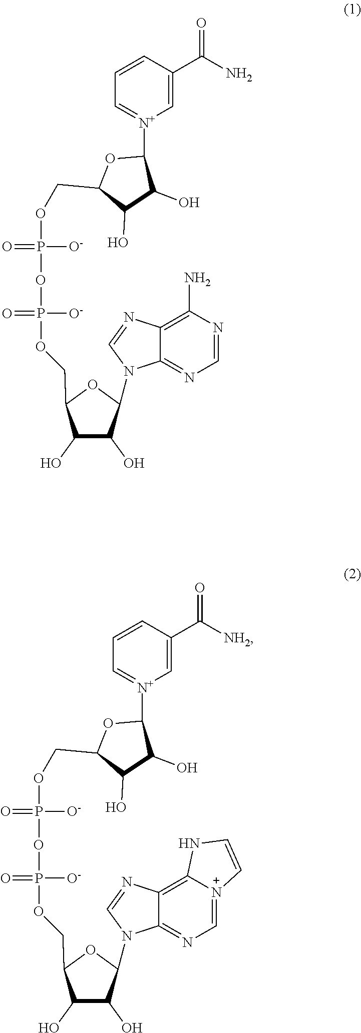

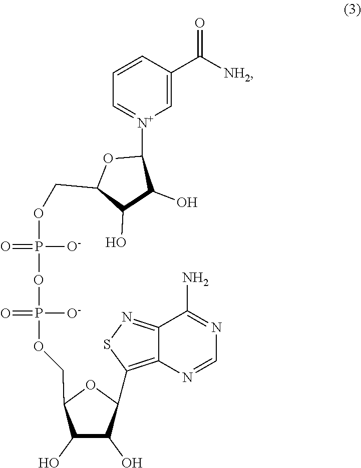

or a salt thereof.

In some illustrative embodiments, this disclosure relates to an assay kit for real-time monitoring protein ubiquitination and/or for identifying an inhibitor for protein ubiquitination as disclosed herein, wherein said synthetic substrate mimic is

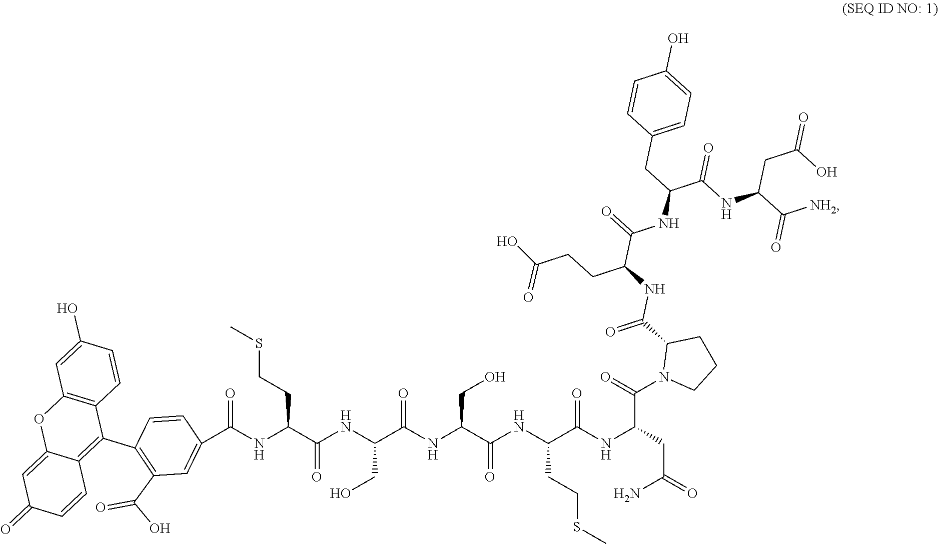 [SMILES: CSCC[C@H](NC(=O)c1ccc(-c2c3ccc(=O)cc-3oc3cc(O)ccc23)c(C(=O)O)c1)C(=O)N[C@@H](CO)C(=O)N[C@@H](CO)C(=O)N[C@@H](CCSC)C(=O)N[C@@H](CC(N)=O)C(=O)N1CCC[C@H]1C(=O)N[C@@H](CCC(=O)O)C(=O)N[C@@H](Cc1ccc(O)cc1)C(=O)N[C@@H](CC(=O)O)C(N)=O]

or a salt thereof.

In some illustrative embodiments, this disclosure relates to an assay kit for real-time monitoring protein ubiquitination and/or for identifying an inhibitor for protein ubiquitination as disclosed herein, wherein said assay system is configured for high-throughput screening for ubiquitination inhibitors.

In some illustrative embodiments, this disclosure relates to an assay kit for real-time monitoring protein ubiquitination and/or for identifying an inhibitor for protein ubiquitination as disclosed herein, wherein said assay system is configured for monitoring the reverse process of protein ubiquitination.

In some illustrative embodiments, this disclosure relates to an assay method for real-time monitoring protein ubiquitination and/or identifying an inhibitor for protein ubiquitination comprising
a. a fluorescent NAD⁺ analogue;
b. a synthetic substrate mimic or a target compound as said inhibitor for protein ubiquitination; and
c. an enzyme of the SidE family for catalyzing ubiquitination.

In some illustrative embodiments, this disclosure relates to an assay method for real-time monitoring protein ubiquitination and/or identifying an inhibitor for protein ubiquitination further comprising 50 mM Tris pH 7.4, 100 mM NaCl, 1 mg/mL BSA, and NAD+ as a control.

In some illustrative embodiments, this disclosure relates to an assay method for real-time monitoring protein ubiquitination and/or identifying an inhibitor for protein ubiquitination as disclosed herein, wherein said fluorescent NAD+ analogue is

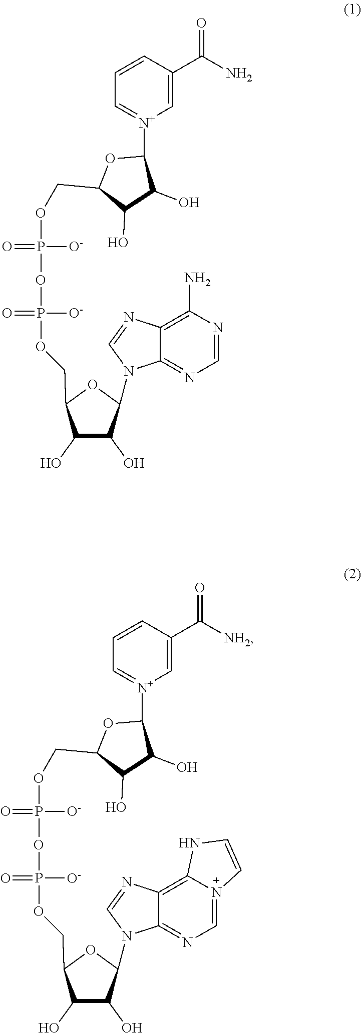

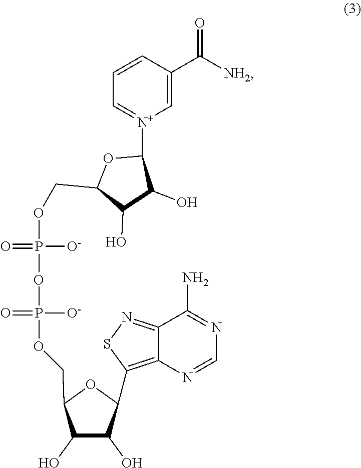

or a salt thereof.

In some illustrative embodiments, this disclosure relates to an assay method for real-time monitoring protein ubiquitination and/or identifying an inhibitor for protein ubiquitination as disclosed herein, wherein said synthetic substrate mimic is

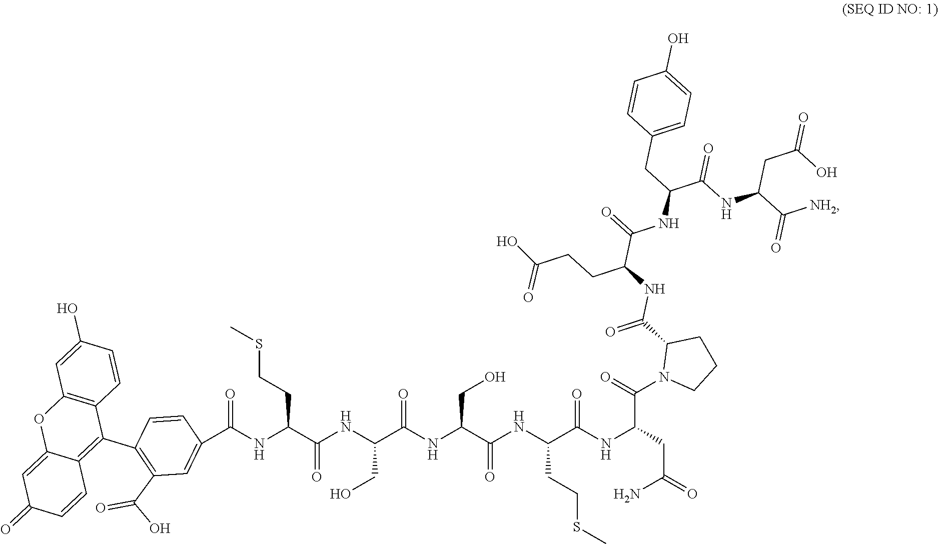 [SMILES: CSCC[C@H](NC(=O)c1ccc(-c2c3ccc(=O)cc-3oc3cc(O)ccc23)c(C(=O)O)c1)C(=O)N[C@@H](CO)C(=O)N[C@@H](CO)C(=O)N[C@@H](CCSC)C(=O)N[C@@H](CC(N)=O)C(=O)N1CCC[C@H]1C(=O)N[C@@H](CCC(=O)O)C(=O)N[C@@H](Cc1ccc(O)cc1)C(=O)N[C@@H](CC(=O)O)C(N)=O]

or a salt thereof.

In some illustrative embodiments, this disclosure relates to an assay method for real-time monitoring protein ubiquitination and/or identifying an inhibitor for protein ubiquitination as disclosed herein, wherein said assay system is configured for high-throughput screening for identifying a ubiquitination inhibitor.

In some illustrative embodiments, this disclosure relates to an assay method for real-time monitoring protein ubiquitination and/or identifying an inhibitor for protein ubiquitination as disclosed herein, wherein assay system is configured for monitoring the reverse process of protein ubiquitination.

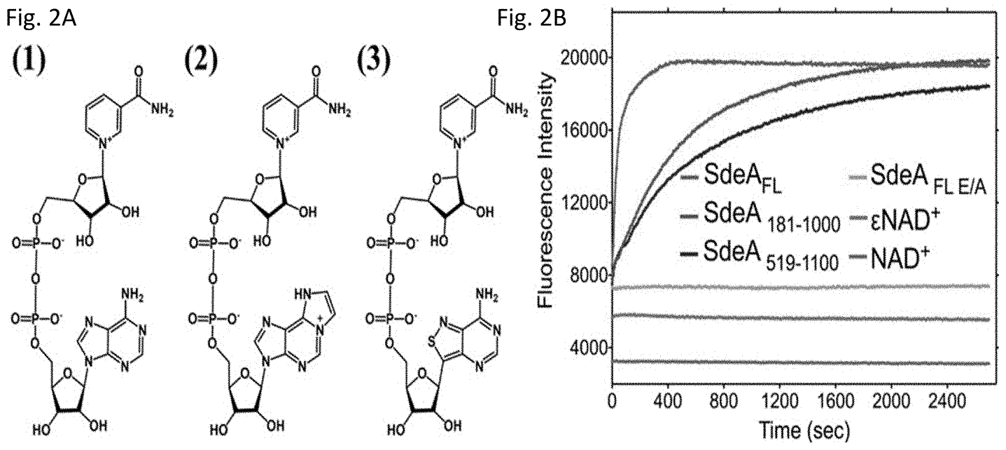

SidE proteins are of great interest to study, especially considering the fact that they are required for optimal Legionella virulence.⁴ The discovery of this new post-translational modification also demands an investigation into whether this process occurs naturally outside of Legionella infection. However, bioinformatics-based identification of homologs has been challenging because of the substantial divergence of the sequence of these effectors from known enzymes. To this end, we have developed continuous, fluorometric assays to measure both steps of this reaction in a sensitive and high-throughput manner. For the first step, we show the utility of two disparate emissive analogues of NAD⁺ (1) in our assays; the classical nicotinamide 1,N6-ethenoadenine dinucleotide (εNAD⁺) (2)¹⁴ as well as the recently-developed N^{tz}AD⁺ (3) which is based on an isothiazolo[3,4-d] pyrimidine core (FIG. 2a).¹⁵ To monitor the crucial second step, we have synthesized a peptide that behaves as a substrate, measuring ubiquitination by fluorescence polarization. These techniques have yielded new insights into the biochemistry of SidE effectors and will prove useful for future attempts of inhibitor screening and discovery/characterization of new members of this enzyme class.

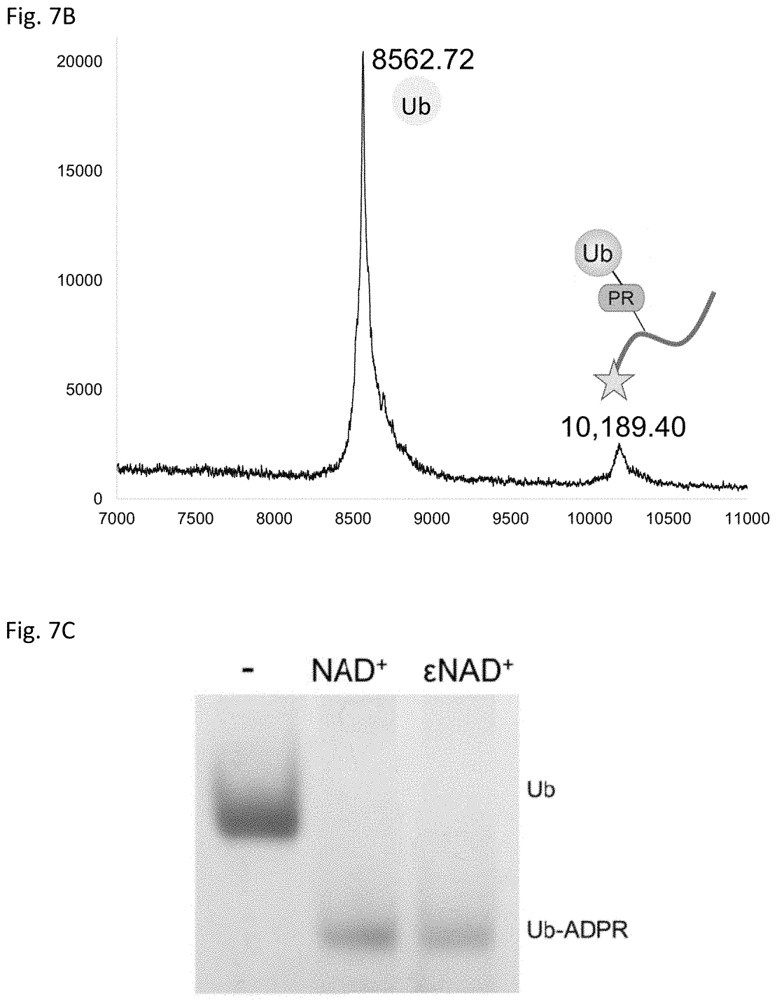

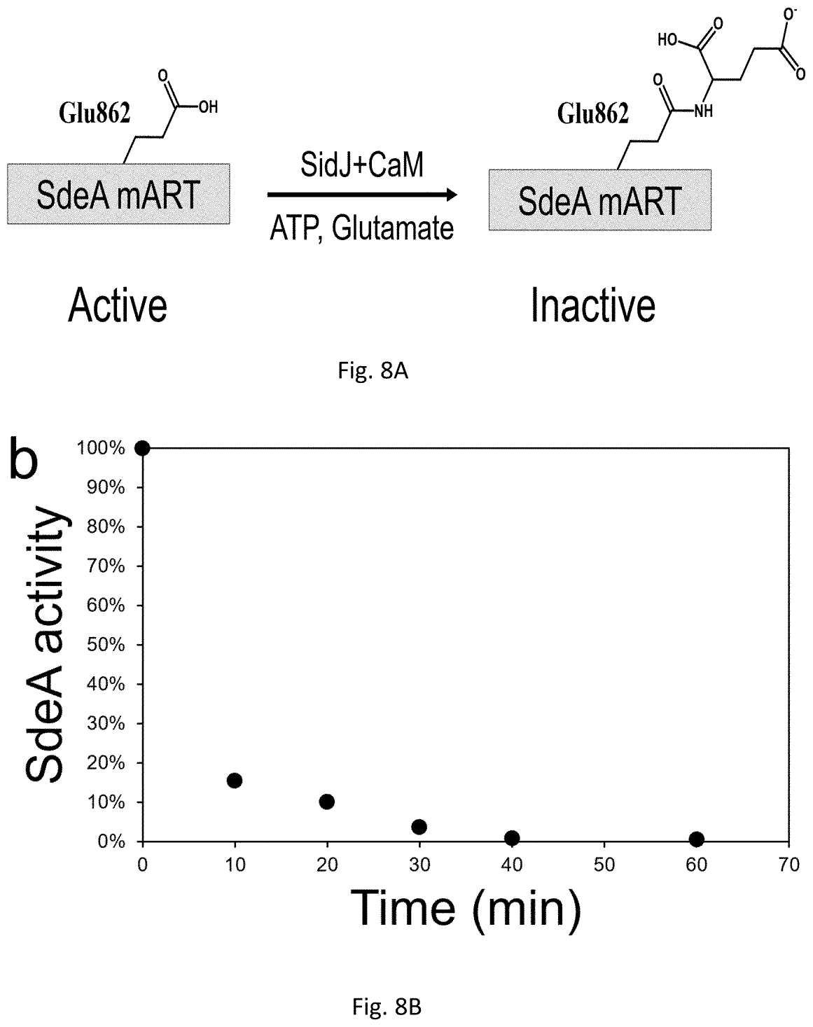

It has been shown that the activity of canonical mART enzymes can be measured with the aforementioned NAD⁺ analogues, due to the fact that these molecules are internally quenched by the nicotinamide moiety.¹⁶ A loss of the nicotinamide from εNAD⁺ or N^{tz}AD⁺ resulting from mART activity will therefore result in a conspicuous increase in fluorescence. In order to test whether the ADP-ribosylation of Ub can be similarly measured, we performed this assay utilizing four constructs of SdeA, a representative SidE family protein. We used the full-length protein (SdeA_{FL}), the ubiquitinating construct spanning residues 181-1000 (SdeA₁₈₁₋₁₀₀₀), the mART construct (SdeA₅₁₉₋₁₁₀₀), and a mutant of the full-length protein unable to perform the mART reaction with catalytic residues Glu860 and Glu862 were mutated to Ala (SdeA_{E/A}). Incubation with εNAD⁺ and Ub resulted in an increase in fluorescence emission at 410 nm, observable in real time (FIG. 2b). Interestingly, we observed that SdeA_{FL} was the most active construct of the three, and SdeA₅₁₉₋₁₁₀₀ was the least active. This effect is in line with previous studies¹⁷ and is possibly a result of the coiled-coil (CC) domain stabilizing the productive orientation of the mART domain. These results were also observed when N^{tz}AD⁺ was used (FIG. 6). The fluorogenic intermediate Ub-EADPR was also isolated and analyzed by LC-MS to verify its identity (FIGS. 7A-7B). Together, these data demonstrate the utility of fluorescent NAD⁺ analogues in studying the first step of SidE-catalyzed ubiquitination.

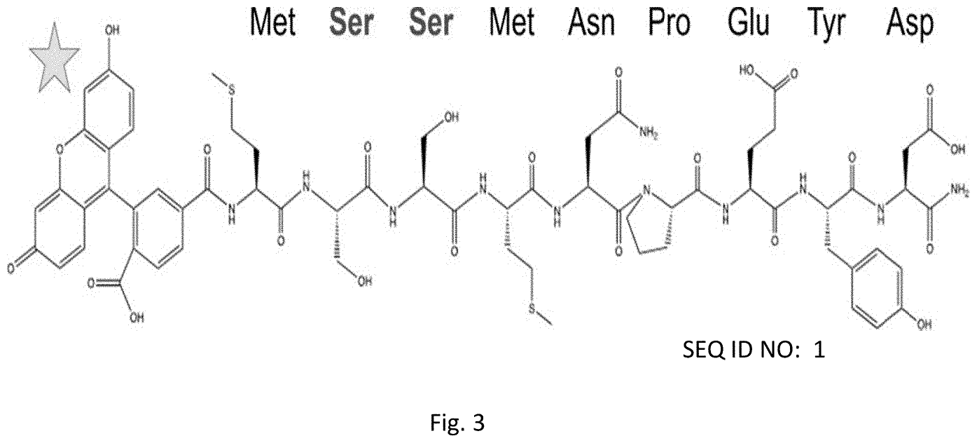

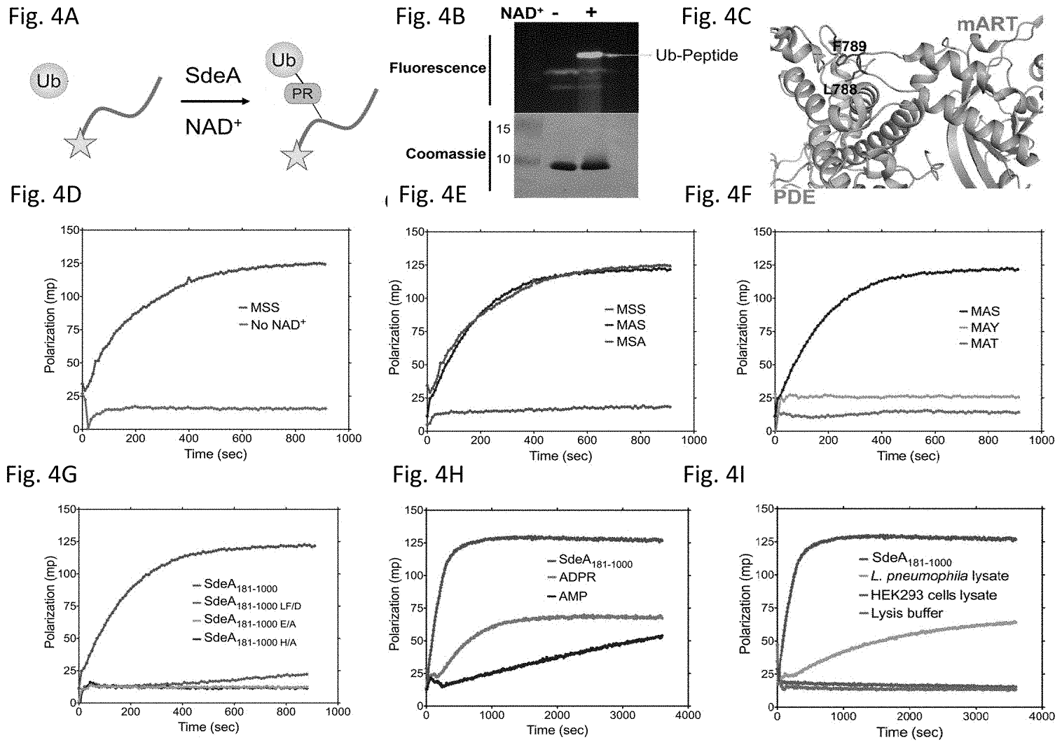

While the above assay is useful for monitoring ADP-ribosylation of Ub, it is limited due to the fact that the second step of the reaction, substrate serine ubiquitination, cannot be tracked with this method. Indeed, further studies have identified substrate ubiquitination as the key step pertinent to Legionella pathogenesis.¹⁷ A mutant of SdeA deficient in catalyzing Step 2 while still capable of catalyzing Step 1 was unable to rescue normal growth in a SidE-deficient strain. Thus, it is important to assay this process quantitatively and develop probes to further understand the mechanism and substrate selection of these enzymes. To this end, a model substrate was synthesized. Previous studies have suggested that SidE proteins target serine residues on unstructured, flexible regions of proteins, such as the N-terminus of many Rab GTPases.¹⁸ We therefore synthesized a peptide consisting of the first 9 residues of Rab1, a known SidE substrate with a 5′-fluorescein group on the amino terminus. These N-terminal residues are unresolved in the crystal structure of Rab1, indicating that they are likely unstructured (FIG. 3). To confirm that this peptide behaved as a ubiquitination substrate, SDS-PAGE analysis revealed the presence of a fluorescent band around 10 kDa upon reaction with Ub, SdeA, and NAD⁺. In the absence of NAD⁺, this band was not observed (FIG. 4b).

We then attempted to track the ubiquitination of the peptide by SdeA in real-time. Due to the approximately 10-fold size difference between the peptide and the peptide-Ub conjugate, we anticipated a fluorescence polarization (FP) increase as the peptide was ubiquitinated by SdeA. When a reaction containing SdeA₁₈₁₋₁₀₀₀, Ub, NAD⁺, and peptide was subjected to FP measurement (λₑₓ 485 nm, λₑₘ 528 nm), the conversion of peptide to Ub-peptide was observed via a significant increase in FP as expected (FIGS. 4, 7B). A Michaelis-Menten analysis with respect to the peptide produces an apparent K_{M} of ˜80 μM, and kcal of ˜1.6 s⁻¹.

Because our fluorescent peptide was derived from Rab1, it contained two Ser residues at positions 2 and 3, respectively. In order to determine whether both residues or only one were targeted by SdeA, analogous peptides were synthesized with the respective Ser residue mutated to Ala (Peptides MSA and MAS). Intriguingly, we found that while peptide MAS retained similar activity to the original peptide (MSS), peptide MSA was not ubiquitinated by SdeA. To further explore the substrate selectivity of SdeA toward other hydroxyl-containing residues, an additional two peptides were synthesized with Thr or Tyr at position 3 (Peptides MAT and MAY). Neither peptide was found to be ubiquitinated by SdeA (FIG. 4f). This result suggests that SidE enzymes recognize serine specifically, and that the positioning of Ser plays a role in substrate recognition. Further structural studies may provide additional insight into the basis of this selectivity. Due to the importance of the SidE family of enzymes in Legionella virulence, and their conservation among a wide variety of Legionella species, they may comprise a new therapeutic target.

The aforementioned assay is a facile technique to screen and characterize inhibitors for this new enzyme class. A previous study suggested that adenosine monophosphate (AMP) could serve as a weak inhibitor of SdeA-catalyzed ubiquitination.¹⁷ Utilizing our assay, incubation with AMP or ADP-ribose resulted in impairment of peptide ubiquitination (FIG. 4h).

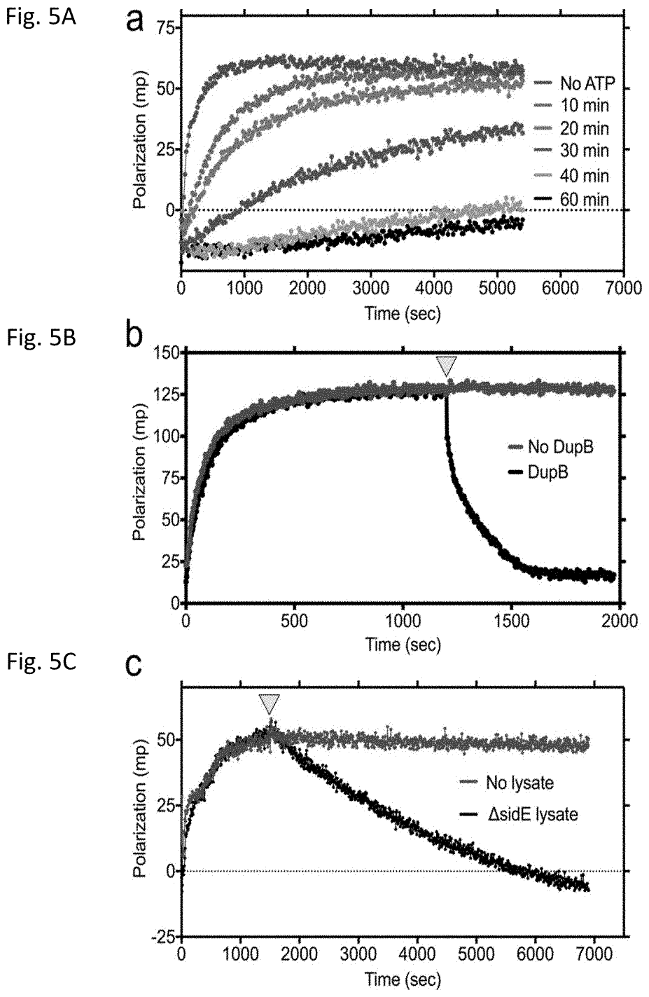

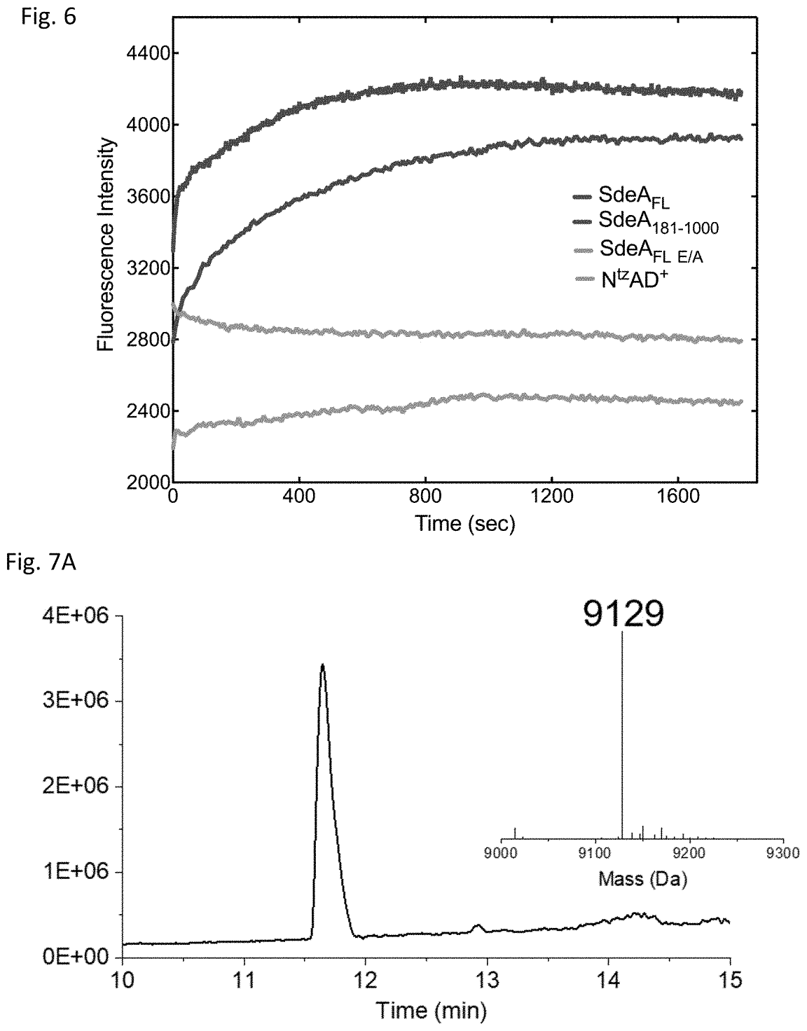

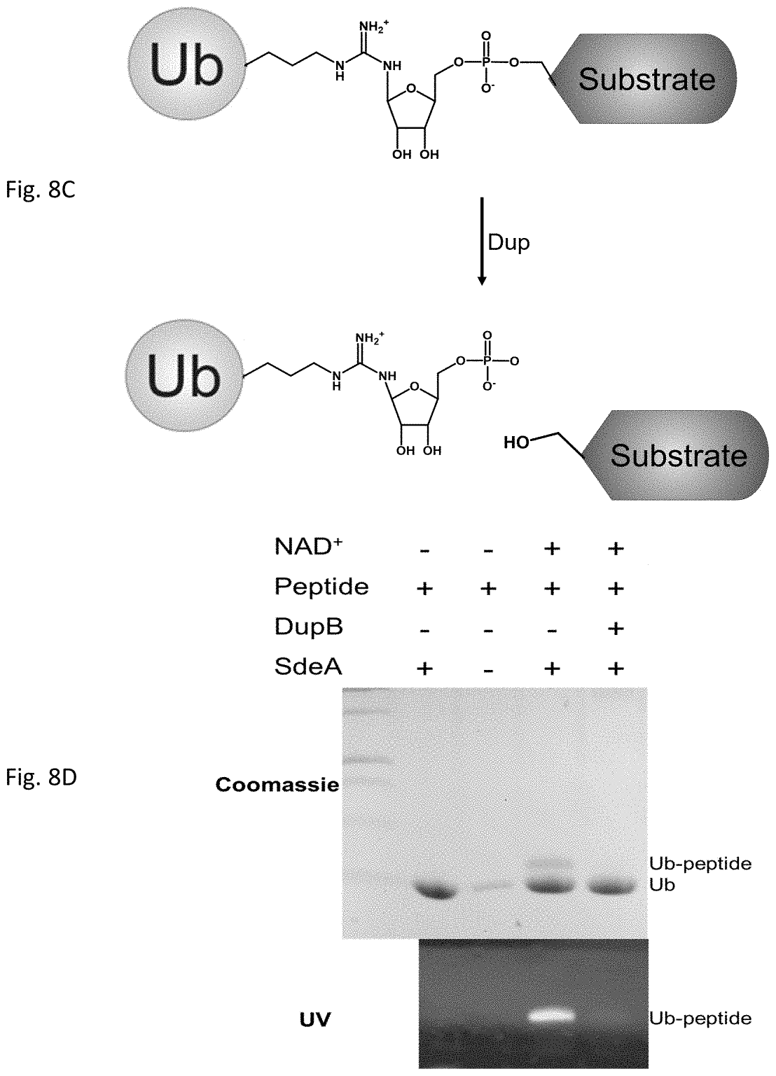

The regulation of phosphoribosyl-ubiquitination (PR-ubiquitination) by other Legionella effectors has attracted considerable recent interest. In order to control the levels of PR-linked ubiquitin, at least two systems have recently been discovered. First, the enzyme SidJ is a glutamylase that covalently modifies the catalytic E860 of SdeA, effectively switching it off.⁸⁻¹¹ The inhibitory effect of SidJ is manifest only when it is bound to the host calmodulin. To study the activity of SidJ, we reacted SdeA with the SidJ-calmodulin complex and observed a striking, time-dependent decrease in SdeA ubiquitinating activity (FIGS. 5A, 8A). By plotting initial rates of our SdeA samples over time, we were able to generate a progress curve for SidJ-catalyzed modification of SdeA (FIG. 8B).

Furthermore, the recently-reported discovery of Legionella enzymes that reverse PR-linked serine ubiquitination catalyzed by the SidE family has intriguingly added a new layer of regulation to this post-translational modification. These enzymes, named DupA and DupB (from lpg2154 and lpg2509, respectively), remove phosphosibosyl-linked ubiquitin from substrates (FIGS. 8C and 8D).^{12,13} Their catalytic action as well as their structure closely resemble that of the PDE domain of SidE proteins. It is possible that these regulatory effectors exist to prevent uncontrolled ubiquitination of substrates by SidE, as deletion of DupA and DupB caused the accumulation of PR-ubiquitinated proteins in infected cells.^{12,13} We tested whether we can monitor the deubiquitination of PR-linked ubiquitin from our synthetic fluorogenic peptide substrate. A two-step assay was performed where peptide ubiquitination was followed by incubation with DupB (also known as SdeD). An increase in FP followed by a marked decrease to baseline levels upon DupB addition was observed (FIG. 5b). Also, in the place of purified DupB, incubation with the lysate of a strain of Legionella pneumophila lacking the SidE family also resulted in a decrease to baseline levels of FP (FIG. 5c). This further highlights the application of this assay towards probing cell lysates to study regulators of SidE enzymes.

The study of serine ubiquitination catalyzed by the SidE family of bacterial effectors has highlighted an elegant new mechanism of post-translationally modifying host proteins. We have developed a robust, real-time method for studying this process via fluorescence, including mutation and inhibition studies that have previously been done via gel electrophoresis-based endpoint analysis. We have also elucidated the position and residue selectivity of SidE enzymes, where future work will be necessary to determine the structural basis for serine and positional preference. Further, it remains to be seen whether serine ubiquitination via the SidE mechanism exists in organisms outside of Legionella. Our method provides a useful tool for discovering new enzymes that can either catalyze or regulate this process.

Parts of this disclosure have been published Kedar Puvar, et al., “Fluorescent Probes for Monitoring Serine Ubiquitination”, Biochemistry 2020, 59, 1309-1313, the contents of which is incorporated herein by reference in their entirety.

Materials and Methods

Synthesis of NAD+ Analogues

εNAD+ was prepared as described.1 Briefly, NAD+ was reacted in aqueous chloroacetaldehyde over a period of 3-4 days until UV absorbance held consistent over time, then purified via ion-exchange chromatography and confirmed by melting-point analysis.

NtzAD+ was prepared as described.2 Briefly, tzAMP was reacted with activated β-nicotinamide mononucleotide, forming the desired product.

Synthesis of Fluorescein-Labeled Rab1 Peptides

Materials:

Fmoc-protected amino acids Fmoc-Met-OH, Fmoc-Ser(tBu)-OH, Fmoc-Asn(Trt)-OH, Fmoc-Pro-OH, Fmoc-Glu(OtBu)-OH, Fmoc-Tyr(tBu)-OH, Fmoc-Asp(OtBu), and Fmoc-Ala-OH, and activation agent HBTU (O-benzotriazole-N,N,N′,N′-tetramethyluronium-hexafluoro-phosphate) were purchased from ChemPep, Inc. (Wellington, Fla., USA). ChemMatrix Rink Amide resin was purchased from Gyros Protein Technologies (Uppsala, Sweden). NHS-Fluorescein was purchased from Thermo-Fisher Scientific (Waltham, Mass., USA). N,N-Dimethylformamide (DMF), Dichloromethane (DCM), Methanol (MeOH), Diisopropylethylamine (DIEA), Trifluoroacetic acid (TFA), Triisopropylsilane (TIPS), and diethyl ether were purchased from Sigma-Aldrich (St. Louis, Mo., USA). Reagents purchased from commercial sources were used without further purification.

Methods:

Peptides were synthesized using standard Fmoc (Fluorenylmethyloxycarbonyl) based solid-phase peptide synthesis on ChemMatrix Rink Amide resin (100 mg, 45 μM). The resin was added to a peptide synthesis flask and treated with the desired Fmoc-protected amino acid (6 eq., 270 μM), HBTU (6 eq., 270 μM) and DIEA (12 eq., 540 μM) in DMF. This mixture was then agitated for 45 minutes at room temperature. The solution was drained from the synthesis flask and the resin was washed with DMF, DCM, MeOH, DCM, and DMF (2×8 mL each). Next, a 20% Piperidine solution in DMF (8 mL) was added to the resin which was agitated for 20 minutes to remove the Fmoc protecting group. The piperidine solution was drained and the resin was washed with DMF, DCM, MeOH, DMC, DMF (2×8 mL each). This process was repeated until all the amino acids in the peptide were coupled on the resin. After the last amino acid deprotection, fluorescein was added to the N-terminus by mixing the resin with NHS-Fluorescein (1.1 eq., 49.5 μM, Fisher Scientific) and DIEA (2.2 eq., 99 μM) in DMF for 12 hours.

The peptides were then cleaved from the solid support by adding a solution of 95% TFA, 2.5% TIPS, and 2.5% H₂O (15 mL). The filtrate was collected, and the resin was washed with TFA (2×15 mL) and DCM (2×15 mL). The filtrates were combined, and the solvents were removed under reduced pressure. The peptide was precipitated using cold diethyl ether and collected via centrifugation. The pellet was dried under reduced pressure and the crude mass was determined. The peptide was then re-suspended in DMSO (10 mg/mL) and purified to homogeneity by reverse phase (RP) HPLC on a Luna C18 semi-prep column using a 60-minute linear solvent gradient. The masses of the pure peptides were obtained using MALDI-ToF mass spectrometry. The concentrations of the peptides were determined using a UV-Vis spectrophotometer.

A calibration curve of the fluorescence intensity (485 nm excitation, 510 nm emission) of fluorescein standard solutions (5-200 μM) was used to determine peptide concentration.

Protein Purification

SdeAFL, SdeA181-1000, SdeA519-1100, SdeD (DupB), and SidJ were cloned into pGEX-6P-1 vector and transformed into E. coli Rosetta™ (DE3) cells to be expressed as GST-tagged proteins. SdeAFL and SdeA181-1000 mutants were generated by site-directed mutagenesis, sequences were confirmed by DNA sequencing and recombinant vectors were transformed into E. coli BL21 (DE3) cells. Ubiquitin cloned into pRSET was transformed into E. coli Rosetta™ (DE3) cells.

Human calmodulin (CaM) was received as a generous gift from Dr. Mark Wilson (University of Nebraska)

Protein expression was carried out by adding an overnight culture of E. coli cells harboring the appropriate recombinant vector into LB medium supplied with 100 μg/ml ampicillin. Cultures were grown in a shaker incubator at 37° C. until reaching an OD600 of 0.5-0.6. Protein expression was induced by adding 0.3 mM IPTG (isopropyl thio-D-galactopyranoside) and cultures were then incubated for 16-18 h at 18° C. Cells were harvested by centrifugation at 6,000×g for 6 min at 4° C. and resuspended in (1×PBS pH 7.4, 400 mM KCl) buffer supplied with lysozyme. Resuspended cells were lysed with French press (Thermo Scientific) and then clarified by ultracentrifugation (Beckman Coulter) at 100,000×g for 1 h at 4° C.

GST-tagged proteins were purified with GST-Sepharose beads (GE Healthcare). Purified proteins were supplied with PreScission™ Protease to cleave the GST tag and dialyzed overnight in (1×PBS pH 7.4, 400 mM KCl, 1 mM DTT) buffer. Dialyzed proteins were then added to GST-Sepharose beads to remove free GST. Proteins were concentrated using Amicon Ultra-15 30K centrifugal columns (Millipore) and protein concentration was determined using NanoDrop A280 (Thermo Scientific).

For ubiquitin purification, cation exchange chromatography was utilized using Mono S beads (GE Healthcare). Clarified lysate produced by ultracentrifugation was boiled in a water bath for few minutes and then centrifuged at 3500 rpm for 10 min to remove protein aggregates. The supernatant was buffer exchanged in Mono S buffer A (50 mM sodium acetate pH 4.5) using Amicon Ultra-15 3K centrifugal columns (Millipore) and then loaded onto the Mono S beads. Ubiquitin was eluted using a gradient elution of Mono S buffer A and B (50 mM sodium acetate pH 4.5, 1 M NaCl). Eluted protein was concentrated using Amicon Ultra-15 3K centrifugal columns (Millipore) and protein concentration was determined using BCA assay.

Purification of εADP-Ribosylated Ubiquitin (Ub-εADPR)

To generate Ub-εADPR, SdeA519-1100 was incubated with Ub and εNAD+ and reacted for 3 hours at 25° C. Reaction mixture was separated by size exclusion and fluorescent fractions corresponding to Ub-εADPR were collected.

Monitoring Ubiquitin ADP-Ribosylation Using NAD+ Analogues

Assays were conducted by mixing 100 μM ubiquitin and 100 μM NAD+, εNAD+ or NtzAD+ in (50 mM Tris pH 7.4, 100 mM NaCl, 1 mg/ml BSA) buffer. Reaction mixtures were left to equilibrate at room temperature for 3 minutes before taking measurements. Reactions were then started by adding 0.5 μM enzyme. Fluorescence intensity was measured using Cytation Multi-Mode Plate Reader (BioTek) at 300 nm excitation and 410 nm emission for εNAD+ assays, and at 338 nm excitation and 410 nm emission for NtzAD+ assays. All assays were performed at least in triplicate.

To compare the ADP-ribosylating activity of SdeA with either NAD+ or εNAD+, an assay was conducted by mixing 100 μM ubiquitin and 100 μM NAD+, εNAD+, or no nucleotide in assay buffer (50 mM Tris pH 7.4, 100 mM NaCl). Reaction mixtures were initiated with addition of 0.5 μM SdeA181-1000 H284A. Reactions were then subjected to native-PAGE analysis and stained with Coomassie Blue.

Rab1 Peptide Ubiquitination In-Gel Fluorescence Assay

5 μM SdeA181-1000, 100 μM ubiquitin and 20 μM fluorescein-labeled peptides were incubated in the presence or absence of 100 μm NAD+ for 10 min at room temperature. Reactions were quenched by 5×SDS/PAGE loading buffer and analyzed by SDS-PAGE. In-gel fluorescence was detected using Azure c600 gel imaging system.

Fluorescence Polarization Assays

Assays were performed by mixing 0.25 μM enzyme, 100 μM ubiquitin and 10 μM fluorescein-labeled peptide in (50 mM Tris pH 7.4, 100 mM NaCl, 1 mg/ml BSA) buffer. Reaction mixtures were left to equilibrate at room temperature for 3 minutes before taking measurements. Reactions were then started by adding 100 μM NAD+.

Fluorescence polarization was measured using a Cytation Multi-Mode Plate Reader (BioTek) using 485 nm excitation and 528 nm emission filters. For lysate assays, the reaction mixture consisted of Legionella pneumophila or HEK293 cells lysate, 100 μM ubiquitin, 100 μM NAD+ and 10 μM fluorescein-labeled MAS peptide.

Michaelis-Menten kinetic analysis was conducted under the assay conditions above, where the concentration of peptide was varied (5-40 μM). The slope of the progress curve was used to determine initial reaction velocity. Initial velocity was plotted against substrate concentration and fit to the Michaelis-Menten equation.

Inhibition assays were conducted by first incubating 0.25 μM SdeA181-1000 with 5 mM ADPR or AMP for 30 min on ice in (50 mM Tris pH 7.4, 100 mM NaCl, 1 mg/ml BSA) buffer. Fluorescence polarization was then measured after adding ubiquitin to 100 μm, NAD+ to 100 μm and fluorescein-labeled MAS peptide to 10 μm to the reaction mixtures.

SidJ assays were performed by mixing 1 μM SidJ, 5 μM SdeA181-1000, 5 μM calmodulin, 500 μm L-glutamate, 5 mM MgCl2 and 1 mM ATP in (50 mM Tris pH 7.4, 100 mM NaCl,

1 mg/ml BSA) buffer. Reaction mixtures were incubated at 37° C. for 10, 20, 30, 40 and 60 min. SidJ activity was quenched by adding 40 mM EDTA at each time point. The quenched reactions were then mixed with 100 μM ubiquitin, 100 μM NAD+ and 10 μM fluorescein-labeled MAS peptide.

Deubiquitination of PR-linked ubiquitin was monitored by performing a two-step assay. First, the ubiqutination reaction was accomplished using SdeA181-1000 and MAS peptide as described previously. This was followed by adding DupB to a final concentration of 6

μm, or Legionella pneumophila lysate lacking the SidE family (ASidE) to reverse the ubiquitination reaction. Fluorescence polarization for SidJ and deubiquitination assays was measured using a Synergy H1 Multi-Mode Plate Reader (BioTek). A sample of this reaction was also utilized for SDS-PAGE gel electrophoresis.

All aforementioned assays were carried out at least in triplicate, at a final reaction volume of 100 μL, and utilizing Grenier 96 well plates (Product no. 655097).

MS Analyses

LC-MS analysis of Ub-εADPR was performed by a Halo™ ES-C18 reverse phase chromatographic column (1.0 mm*15 cm, 3.4 μm particle size) (Advanced Materials Technology) for top-down LC-MS separation. An Accela UHPLC system (Thermo) coupled with an LTQ Velos mass spectrometer (Thermo) was used to perform sample injection, gradient elution and mass characterization. Spectra were deconvoluted using Magtran software.

Mass spectra of the nucleotide analogues were analyzed using a Waters SQD2 mass spectrometer coupled to a Waters Acquity UPLC system.

To determine the MS of the ubiquitinated MAS Rab1 peptide, an ubiquitination reaction conducted using identical conditions as described in the FP assay above was subjected to SDS-PAGE gel electrophoresis. The fluorescent band, corresponding to Ub-PR-peptide was excised and washed with 50% acetonitrile for 10 minutes. Protein was extracted by overnight shaking in a solution of gel extraction solution (1:3:2 formic acid/water/2-propanol). MS was recorded using a Voyager DE Pro MALDI-ToF mass spectrometer (Applied Biosystems).

Additional disclosure is found in Appendix-A, filed herewith, entirety of which is incorporated herein by reference into the present disclosure.

Those skilled in the art will recognize that numerous modifications can be made to the specific implementations described above. The implementations should not be limited to the particular limitations described. Other implementations may be possible.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES CITED

(1) Komander, D.; Rape, M. The Ubiquitin Code. Annu. Rev. Biochem. 2012, 81 (1), 203-229. https://doi.org/10.1146/annurev-biochem-060310-170328.
(2) Ashida, H.; Kim, M.; Sasakawa, C. Exploitation of the Host Ubiquitin System by Human Bacterial Pathogens. Nat. Rev. Microbiol. 2014, 12 (6), 399-413. https://doi.org/10.1038/nrmicro3259.
(3) Qiu, J.; Luo, Z.-Q. Legionella and Coxiella Effectors: Strength in Diversity and Activity. Nat. Rev. Microbiol. 2017, 15 (10), 591-605. https://doi.org/10.1038/nrmicro.2017.67.
(4) Qiu, J.; Sheedlo, M. J.; Yu, K.; Tan, Y.; Nakayasu, E. S.; Das, C.; Liu, X.; Luo, Z.-Q. Ubiquitination Independent of E1 and E2 Enzymes by Bacterial Effectors. Nature 2016, 533 (7601), 120-124. https://doi.org/10.1038/nature17657.
(5) Bhogaraju, S.; Kalayil, S.; Liu, Y.; Bonn, F.; Colby, T.; Matic, I.; Dikic, I. Phosphoribosylation of Ubiquitin Promotes Serine Ubiquitination and Impairs Conventional Ubiquitination. Cell 2016, 167 (6), 1636-1649.e13. https://doi.org/10.1016/j.cell.2016.11.019.
(6) Kotewicz, K. M.; Ramabhadran, V.; Sjoblom, N.; Vogel, J. P.; Haenssler, E.; Zhang, M.; Behringer, J.; Scheck, R. A.; Isberg, R. R. A Single Legionella Effector Catalyzes a Multistep Ubiquitination Pathway to Rearrange Tubular Endoplasmic Reticulum for Replication. Cell Host Microbe 2017, 21 (2), 169-181. https://doi.org/10.1016/j.chom.2016.12.007.
(7) Puvar, K.; Luo, Z.-Q.; Das, C. Uncovering the Structural Basis of a New Twist in Protein Ubiquitination. Trends Biochem. Sci. 2019, 44 (5), 467-477. https://doi.org/10.1016/j.tibs.2018.11.006.
(8) Black, M. H.; Osinski, A.; Gradowski, M.; Servage, K. A.; Pawlowski, K.; Tomchick, D. R.; Tagliabracci, V. S. Bacterial Pseudokinase Catalyzes Protein Polyglutamylation to Inhibit the SidE-Family Ubiquitin Ligases. Science 2019, 364 (6442), 787-792. https://doi.org/10.1126/science.aaw7446.
(9) Gan, N.; Zhen, X.; Liu, Y.; Xu, X.; He, C.; Qiu, J.; Liu, Y.; Fujimoto, G. M.; Nakayasu, E. S.; Zhou, B.; et al. Regulation of Phosphoribosyl Ubiquitination by a Calmodulin-Dependent Glutamylase. Nature 2019, 572 (7769), 387-391. https://doi.org/10.1038/s41586-019-1439-1.
(10) Bhogaraju, S.; Bonn, F.; Mukherjee, R.; Adams, M.; Pfleiderer, M. M.; Galej, W. P.; Matkovic, V.; Lopez-Mosqueda, J.; Kalayil, S.; Shin, D.; et al. Inhibition of Bacterial Ubiquitin Ligases by SidJ-calmodulin Catalysed Glutamylation. Nature 2019, 572 (7769), 382-386. https://doi.org/10.1038/s41586-019-1440-8.
(11) Sulpizio, A.; Minelli, M. E.; Wan, M.; Burrowes, P. D.; Wu, X.; Sanford, E. J.; Shin, J.-H.; Williams, B. C.; Goldberg, M. L.; Smolka, M. B.; et al. Protein Polyglutamylation Catalyzed by the Bacterial Calmodulin-Dependent Pseudokinase SidJ. eLife 2019, 8, e51162. https://doi.org/10.7554/eLife.51162.
(12) Wan, M.; Sulpizio, A. G.; Akturk, A.; Beck, W. H. J.; Lanz, M.; Faça, V. M.; Smolka, M. B.; Vogel, J. P.; Mao, Y. Deubiquitination of Phosphoribosyl-Ubiquitin Conjugates by Phosphodiesterase-Domain-containing Legionella Effectors. Proc. Natl. Acad. Sci. 2019. https://doi.org/10.1073/pnas.1916287116.
(13) Shin, D.; Mukherjee, R.; Liu, Y.; Gonzalez, A.; Bonn, F.; Liu, Y.; Rogov, V. V.; Heinz, M.; Stolz, A.; Hummer, G.; et al. Regulation of Phosphoribosyl-Linked Serine Ubiquitination by Deubiquitinases DupA and DupB. Mol. Cell 2019, 0 (0). https://doi.org/10.1016/j.molcel.2019.10.019.
(14) Barrio, J. R.; Secrist, J. A.; Leonard, N. J. A Fluorescent Analog of Nicotinamide Adenine Dinucleotide. Proc. Natl. Acad. Sci. 1972, 69 (8), 2039-2042. https://doi.org/10.1073/pnas.69.8.2039.
(15) Rovira, A. R.; Fin, A.; Tor, Y. Emissive Synthetic Cofactors: An Isomorphic, Isofunctional, and Responsive NAD+ Analogue. J. Am. Chem. Soc. 2017, 139 (44), 15556-15559. https://doi.org/10.1021/jacs.7b05852.
(16) Klebl, B. M.; Pette, D. A Fluorometric Assay for Measurement of Mono-ADP-Ribosyltransferase Activity. Anal. Biochem. 1996, 239 (2), 145-152. https://doi.org/10.1006/abio.1996.0309.
(17) Kalayil, S.; Bhogaraju, S.; Bonn, F.; Shin, D.; Liu, Y.; Gan, N.; Basquin, J.; Grumati, P.; Luo, Z.-Q.; Dikic, I. Insights into Catalysis and Function of Phosphoribosyl-Linked Serine Ubiquitination. Nature 2018, 557 (7707), 734-738. https://doi.org/10.1038/s41586-018-0145-8.
(18) Wang, Y.; Shi, M.; Feng, H.; Zhu, Y.; Liu, S.; Gao, A.; Gao, P. Structural Insights into Non-Canonical Ubiquitination Catalyzed by SidE. Cell 2018, 173 (5), 1231-1243.e16. https://doi.org/10.1016/j.cell.2018.04.023.

We claim:

1. An assay system for real-time monitoring protein ubiquitination and/or for identifying an inhibitor for protein ubiquitination comprising
a. a fluorescent NAD⁺ analogue;
b. a synthetic substrate mimic or a target compound as said inhibitor;
c. an enzyme of the SidE family for catalyzing ubiquitination.

2. The assay system according to claim 1 further comprising 50 mM Tris pH 7.4, 100 mM NaCl, 1 mg/mL BSA, and NAD⁺ as a control.

3. The assay system according to claim 1, wherein said fluorescent NAD⁺ analogue is 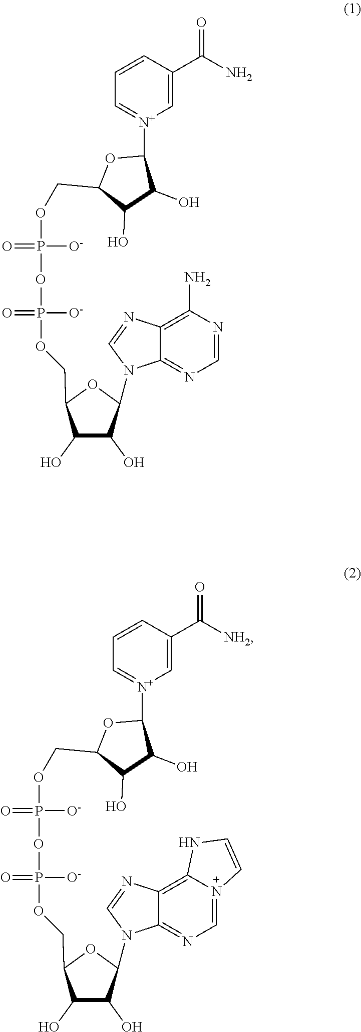 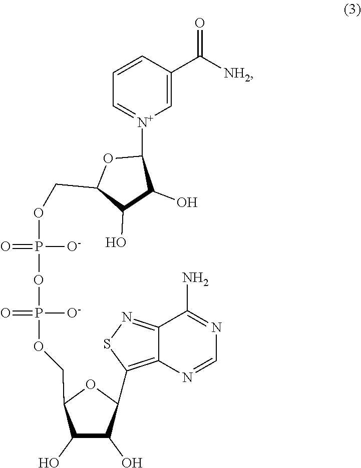
or a salt thereof.

4. The assay system according to claim 1, wherein said synthetic substrate mimic is 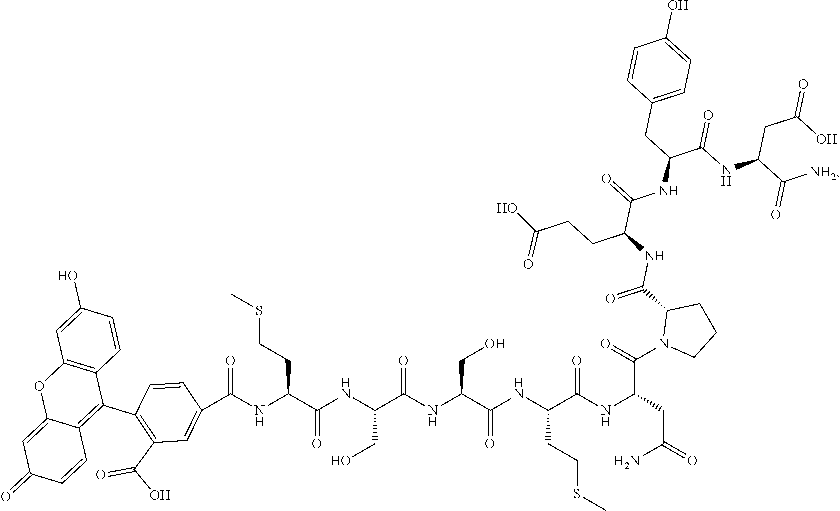 [SMILES: CSCC[C@H](NC(=O)c1ccc(-c2c3ccc(=O)cc-3oc3cc(O)ccc23)c(C(=O)O)c1)C(=O)N[C@@H](CO)C(=O)N[C@@H](CO)C(=O)N[C@@H](CCSC)C(=O)N[C@@H](CC(N)=O)C(=O)N1CCC[C@H]1C(=O)N[C@@H](CCC(=O)O)C(=O)N[C@@H](Cc1ccc(O)cc1)C(=O)N[C@@H](CC(=O)O)C(N)=O]
or a salt thereof (SEQ ID NO: 1).

5. The assay system according to claim 1, wherein said assay system is configured for high-throughput screening for ubiquitination inhibitors.

6. The assay system according to claim 1, wherein said assay system is configured for monitoring the reverse process of protein ubiquitination.

7. An assay kit for real-time monitoring protein ubiquitination and/or for identifying an inhibitor for protein ubiquitination comprising
a. a fluorescent NAD⁺ analogue;
b. a synthetic substrate mimic or a compound as said inhibitor for protein ubiquitination; and
c. an enzyme of the SidE family for catalyzing ubiquitination.

8. The assay kit according to claim 7 further comprising comprising 50 mM Tris pH 7.4, 100 mM NaCl, 1 mg/mL BSA, and NAD+ as a control.

9. The assay kit according to claim 7, wherein said fluorescent NAD+ analogue is 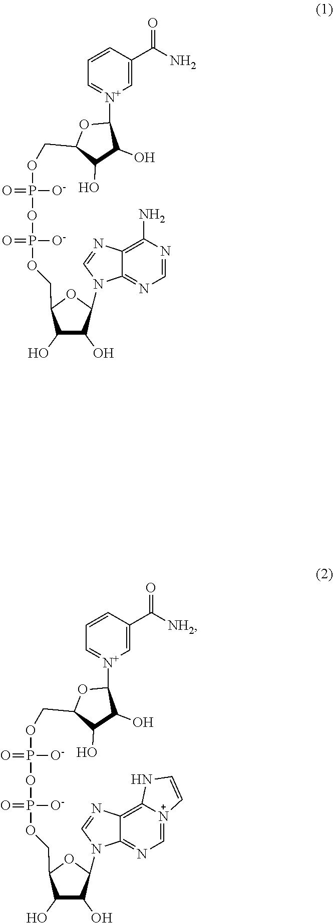 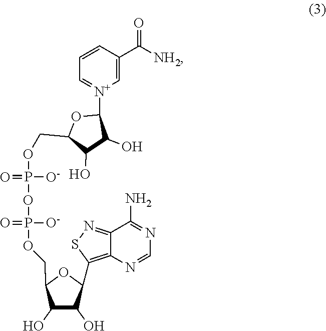
or a salt thereof.

10. The assay kit according to claim 7, wherein said synthetic substrate mimic is 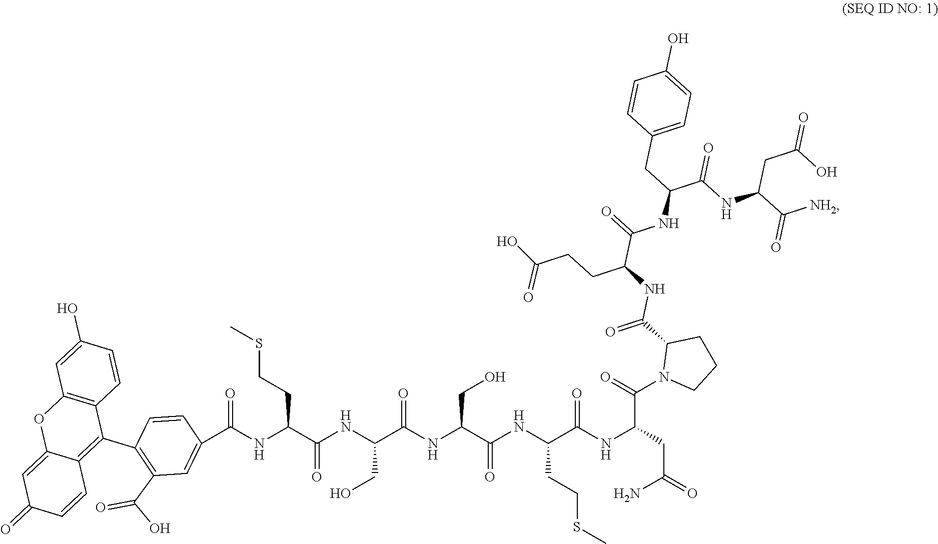 [SMILES: CSCC[C@H](NC(=O)c1ccc(-c2c3ccc(=O)cc-3oc3cc(O)ccc23)c(C(=O)O)c1)C(=O)N[C@@H](CO)C(=O)N[C@@H](CO)C(=O)N[C@@H](CCSC)C(=O)N[C@@H](CC(N)=O)C(=O)N1CCC[C@H]1C(=O)N[C@@H](CCC(=O)O)C(=O)N[C@@H](Cc1ccc(O)cc1)C(=O)N[C@@H](CC(=O)O)C(N)=O]
or a salt thereof.

11. The assay kit according to claim 7, wherein said assay system is configured for high-throughput screening for ubiquitination inhibitors.

12. The assay kit according to claim 7, wherein said assay system is configured for monitoring the reverse process of protein ubiquitination.

13. An assay method for real-time monitoring protein ubiquitination and/or identifying an inhibitor for protein ubiquitination comprising
a. a fluorescent NAD⁺ analogue;
b. a synthetic substrate mimic or a target compound as said inhibitor for protein ubiquitination; and
c. an enzyme of the SidE family for catalyzing ubiquitination.

14. The assay method according to claim 13 further comprising 50 mM Tris pH 7.4, 100 mM NaCl, 1 mg/mL BSA, and NAD+ as a control.

15. The assay method according to claim 13, wherein said fluorescent NAD+ analogue is 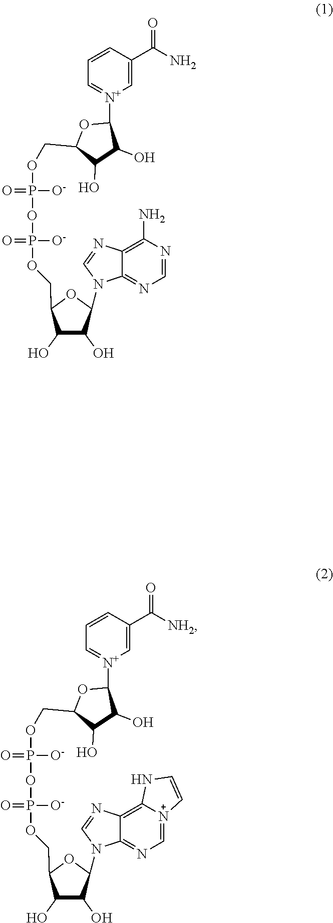 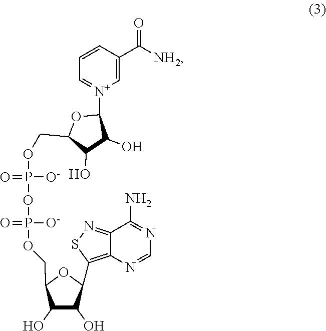
or a salt thereof.

16. The assay method according to claim 13, wherein said synthetic substrate mimic is 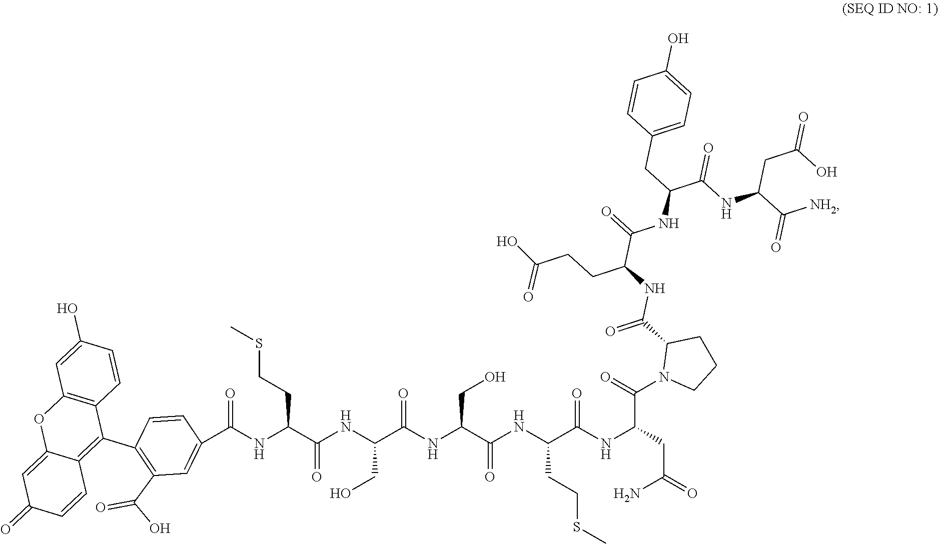 [SMILES: CSCC[C@H](NC(=O)c1ccc(-c2c3ccc(=O)cc-3oc3cc(O)ccc23)c(C(=O)O)c1)C(=O)N[C@@H](CO)C(=O)N[C@@H](CO)C(=O)N[C@@H](CCSC)C(=O)N[C@@H](CC(N)=O)C(=O)N1CCC[C@H]1C(=O)N[C@@H](CCC(=O)O)C(=O)N[C@@H](Cc1ccc(O)cc1)C(=O)N[C@@H](CC(=O)O)C(N)=O]
or a salt thereof.

17. The assay method according to claim 13, wherein said assay system is configured for high-throughput screening for identifying a ubiquitination inhibitor.

18. The assay method according to claim 13, wherein said assay system is configured for monitoring the reverse process of protein ubiquitination.
